(19) United States
(12) Patent Application Publication
(10) Pub. No.: US 2006/0210584 A1
(43) Pub. Date: Sep. 21, 2006

(54) Method for preparing citrinin-free Monascus biomass and use of citrinin-free Monascus biomass

(76) Inventor: Siu Wai Chiu, Hong Kong SAR (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/084,848

(22) Filed: Mar. 18, 2005

(51) Int. Cl. A61K 36/09; C12P 1/00; C12N 1/14

(52) U.S. Cl. 424/195.15; 435/41; 435/254.1

(57) ABSTRACT

Disclosed is a method for preparing a citrinin-free Monascus biomass. Also disclosed are a pharmaceutical and a functional food comprising a preparation by the method. The Monascus biomass possesses anti-oxidation property, and shows anti-proliferation property on cancer cells via apoptosis pathway.

### FIELD OF THE INVENTION

This invention relates to a method for preparing a Monascus biomass and use of the prepared Monascus biomass, particularly relates to a method for preparing a citrinin-free Monascus biomass by a submerged fermentation protocol, and use of the Monascus biomass in preparing functional food and pharmaceuticals.

### BACKGROUND OF THE INVENTION

Functional foods, also known as nutraceuticals, medical foods or nutritional foods, are rooted in the philosophy of Chinese medicated diet. The FDA of US defines “functional foods” as foods that by virtue of physiologically active components provides benefits beyond basic nutrition and may prevent diseases and promote health.

Fungi are biological resources next to plants being popularly exploited in pharmaceutical industry and functional foods. Fungi are an ideal food because they have a fairly high content of proteins (typically 20-30% crude proteins, dry weight basis) containing all of the amino acids which are essential to human and animal nutrition. Fungal foods are characteristically lower in fat, and are virtually free of cholesterol.

In accordance with the Traditional Chinese Medicine (TCM) Standard set forth in Pharmacopoeia of People's Republic of China, red rice (also called Monascus-fermented rice, or red yeast rice) is a TCM. Red rice is also considered as an additive for foods and beverages, and has been widely used in the food processing industry for the production of fermented bean curd, and wine, and for the artificial coloring of meat. Red rice is regarded as a GRAS (generally regarded as safe) product.

It has been known that Monascus-fermented rice functions in invigorating spleen and digestion, promoting blood circulation and resolving blood stasis. Gamma-aminobutyric acid (GABA) existing in Monascus-fermented rice works well in preventing hypertension as a hypotensive agent. Also, GABA that is a natural inhibitory neurotransmitter in the central nervous system and essential for brain metabolism and function in vertebrates improves visual cortical function in senescent monkeys and signalled the nitrogen uptake in plants. This Monascus fermented rice is also accepted as a functional food and sold as dietary supplement worldwide for the fungal metabolite lovastatin, which inhibits cholesterol biosynthesis and benefits patients suffering from cardiovascular diseases. Moreover, Monascus fermented rice is a natural food colorant and preservative with antibacterial properties. Monascus beverages and pigments have been found to have an anti-mutagenic effect and anti-oxidation effect.

Lovastatin is first reported in Monascus ruber and by competitively inhibiting HMG Co-A reductase, leads to reduced conversion of mevalonate in terpenes and sterols but neutral effect on incorporation of acetate into fatty acids. In human consumption, statins are used in cure of hyperlipidemia, anti-inflammation and prevention of acute coronary events in persons with average to high cholesterol levels. Recently lovastatin has been found to show beneficial anti-cancer effect. Commercial sale for Monascus fermented rice or other products is limited or banned by the frequent detection of citrinin, which is also a Monascus metabolite. Citrinin is a nephrotoxin, heptatoxin and carcinogen. It induces DNA breakage and possesses aneuploidogenic and clastogenic potential in cultured cell systems. Fermentation conditions, cultivation media and strains used for cultivation affect the yield of citrinin, but processes for citrinin-free fermentation have never been reported.

Monascus metabolites, such as pigments (conventional red, orange, yellow), enzymes (e.g. phytase), statins, have been targeted, and the optimised conditions for the production of these products have been disclosed. Most interests have been on Monascus pigments exploring their potential uses as food colorants (pigments in food industry) and cosmetic industry, which includes six conventional Monascus pigments as shown in FIG. 1, namely: yellow (monascin and ankaflavin) which are the reduced forms of the two orange pigments, orange (monascorubrin and rubropunctatin), which have different side chain at the ozo-lactone ring, and red (monascorubramine and rubropunctamine), which are nitrogen analogues of the orange pigments.

These pigments are produced mainly in the cell-bound state. They have lower solubilities in water but higher solubilities in fat, are sensitive to heat and pH, and fade with exposure to light. Novel water-soluble Monascus pigment molecules and their optimisation in pigment extractions have been reported. For human food products or animal feed, the production technology is by solid-state-fermentation using solid substrates, rice grains or cereals or wastes from milk industry or concentrated organic wastes, or by semi-solid fermentation using steamed rice. However, in many of these reports, citrinin, the undesirable Monascus metabolite, has also been found. The presence of citrinin becomes the bottleneck in commercialising Monascus products.

The term “fermentation” is derived from a Latin verb fervere, to boil, thus describing the appearance of the action of yeast on extracts of fruit or malted grain. This meaning has been expanded and now describes any process for the production of desirable products by the mass culture of a microorganism. Solid-state-fermentation is applied in production of edible mushrooms. For example, the cultivated mushroom is produced on a mixture of solid substrates: straw and horse manure, and fungal fermented products such as Indonesian tempeh which is a white cake produced by fermentation of partially cooked soybean cotyledons with fungus Rhizopus oligosporus. The raw material for fermentation is solid in nature. Mushroom cultivation is by itself a waste treatment process in bioconverting the abundant solid agricultural and industrial wastes, such as straw, crop residues and sawdust, into commercial mushroom products. In this type of fermentation technology, uniformity in gaseous exchange, heat dispersal, substrate/inoculum mixing, nutrient supplementation and product yield and quality are difficult to control. This is usually a long process taking one or more months to get the products. Production can be carried out in open or enclosed systems. Contamination originated from the environment and operators has to be regularly monitored and safe-guarded. The products are either aerial biomass (e.g. sclerotia, mushrooms with tissue differentiation) or ramified solid substrate with fungal mycelium (e.g. tempeh, a heterogeneous mass).

In contrast, submerged fermentation employs a liquid medium or semi-solid medium for production of desirable products, such as biomass (single cell protein, yeast cells, etc.), and metabolites (antibiotics, organic acids, enzymes, etc.). The environmental fermentation conditions are controlled in terms of illumination, aeration, agitation, and pH. The liquid medium with agitation provides a uniform environment in contrast to the heterogeneity in solid-state-fermentation. The target micro-organism (the producer), usually a fungus or a bacterium, is grown at its optimised conditions to maximise the yield of the desirable products, and an automation system is usually installed to maintain the optimised environmental conditions favouring the microbial physiology for product yield. The whole production is carried out with aseptic technique and is usually in a GMP (good manufacture practice) laboratory. This is an enclosed system avoiding airborne contamination. As the producer is at its optimal physiology in fermentation, the whole fermentation process takes a short period of time in terms of days. Also, a homogenous product is yielded. For example, cells produced are of the same physiology or growth phase.

Submerged fermentation was used to optimally produce Monascus pigments, enzymes and statins. However, there have been no reports developing a novel submerged fermentation system to produce pure a Monascus biomass which is citrinin-free and contains rich sources of amino acids, minerals and dietary fibers, and a balanced profile of saturated and unsaturated fatty acids till now. Also there have been no reports on a Monascus biomass which shows anti-cancer effect via the mechanism of enhancement of apoptosis (programmed cell death) in cancer cells and high antioxidation capacities as well.

### SUMMARY OF THE INVENTION

The present invention modifies in the aspects of fermentation technology and fungal physiology (medium composition and fermentation conditions) to produce pure Monascus biomass which is consistently citrinin-free.

Thus, one aspect of the present invention provides a method for producing a citrinin-free Monascus biomass, which includes:

preparing an inoculum of Monascus purpureus;

inoculating the inoculum into a medium;

fermenting the medium under a submerged condition; and

harvesting the Monascus biomass from the medium.

Another aspect of the invention is to provide a Monascus biomass prepared by a method defined herein.

Another aspect of the present invention is directed to a pharmaceutical which comprises an effective amount of a citrinin-free Monascus biomass prepared by the method disclosed in the invention. The pharmaceutical can treat or prevent a disorder associated with proliferating cancer cells.

Another aspect of the invention is to provide a method for preventing and treating a disorder associated with proliferating cancer cells in a subject, comprising administrating a therapeutically effective amount of a citrinin-free Monascus biomass prepared by the method disclosed in the invention to the subject.

Yet another aspect of the present invention relates to a functional food, comprising a citrinin-free Monascus biomass prepared by the method disclosed in the invention.

The Monascus biomass produced in the invention is red in color and comprises physiologically active components such as lovastatin, GABA, squalene, ergosterol and dietary fiber.

Those skilled in the art may select suitable strains of Monascus anka, Monascus rubber and Monascus purpureus used in the invention. Monascus purpureus strain IM 888 is preferably, but not limited to, used in the invention.

In an embodiment of the method of the invention, the inocucatng is performed under illumination. The first day of fermentation is kept stationary, and a mycelial inoculum is used.

In a preferred embodiment of the invention, a semi-defined broth is used as a medium.

The fermenting of the method in the invention is firstly kept stationary and then shaken, and preferably the fermenting is performed at a condition of aeration and illumination.

In an embodiment of the invention, the cancer cells are of human hepatocellular carcinoma and human colon carcinoma.

### BRIEF DESCRIPTION OF THE DRAWING

FIG. 1 shows the stimulation effect of glutamate on the production of red pigments (conventional red pigments and water-soluble red pigments);

FIG. 2 shows the biosynthetic pathways in Monascus. The structures of anti-oxidant dimerumic acid, cholesterol-lowering agent lovastatin, citrinin and basic skeleton of red Monascus pigments are shown. Red pigments, citrinin and lovastatins are de novo synthesized by the polyketide pathway. Red pigments have N as its component element unlike citrinin and lovastatins. Also, GABA is a glutamate derivative.

FIG. 3 shows a HPLC chromatogram of an alcohol extract of a Monascus biomass prepared by the invention;

FIG. 4 shows the contents of minerals in a Monascus biomass of the invention;

FIG. 5 shows the contents of free amino acids (5a) and total amino acids (5b) of a Monascus biomass of the invention;

FIG. 6 shows the terpene profile in a Monascus biomass of the invention by GC-MS, in which (a) represents squalene and (b) is ergosterol;

FIG. 7 shows the anti-oxidation capacities of a Monascus biomass of the invention in which FIG. 7a shows the scavenging of DPPH radicals, FIG. 7b shows the scavenging of superoxide radicals, FIG. 7c shows the inhibition of lipid peroxidation, and FIG. 7d shows the reducing power of the Monascus biomass;

FIG. 8 shows the anti-proliferation effect of a Monascus biomass of the invention on liver cancer cells, in which FIG. 8a shows relative population size of treated cancer cells with the Monascus biomass via that of a control at a particular day, FIG. 8b shows the decrease in cDNA yield after treatment with the Monascus biomass, FIG. 8c shows the enhanced expression of gene p53 after treatment with the Monascus biomass, and FIG. 8d shows that the Monascus biomass has neutral effect on expression of gene Bax; and

FIG. 9 shows that no citrinin is detected in a Monascus biomass of the invention by GC-SIM, in which FIG. 9a shows authentic standard of citrinin which is broken down into fragmented compounds by high temperature in GC and is resolved into two peaks at SIM mode at 15.5 min and 16.5 min, and FIG. 9b shows a Monascus biomass which does not resolve any peak at the two retention times.

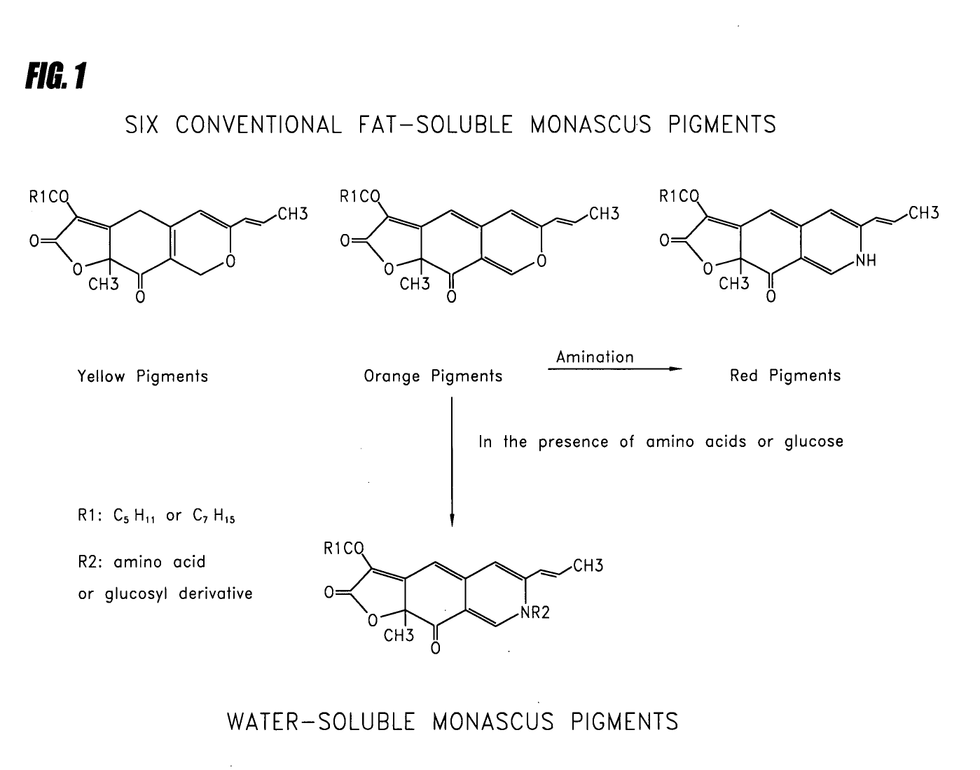

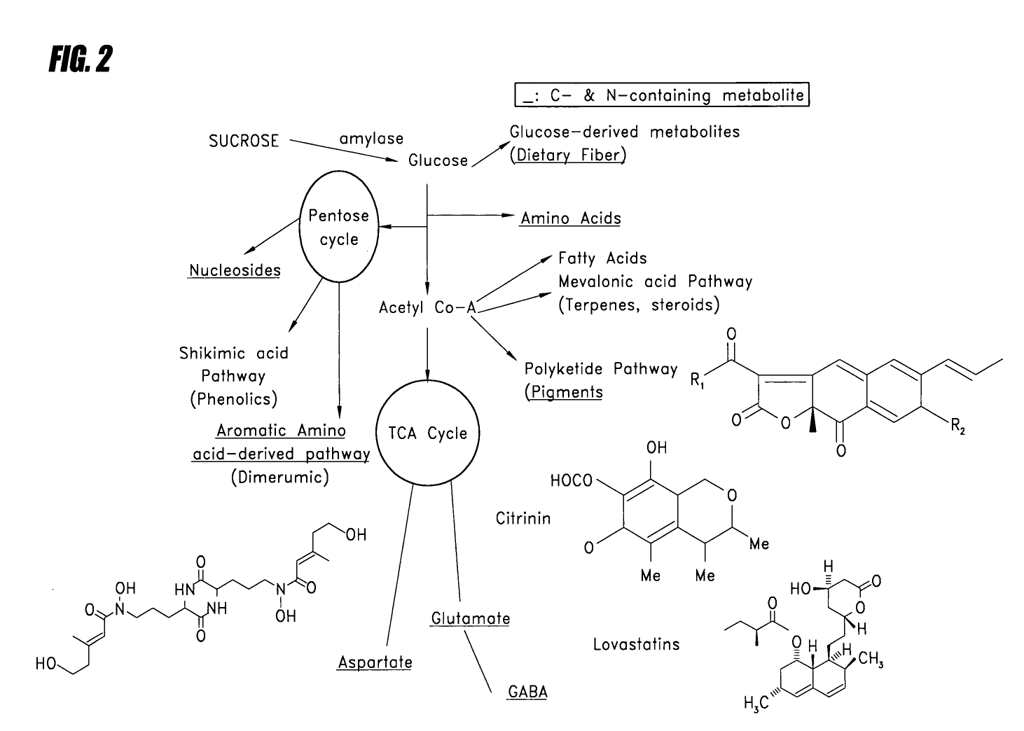

### DETAILED DESCRIPTION OF THE INVENTION

To address deficiencies in the production of Monascus biomass by fermentation, the present invention provides a method to produce pure Monascus biomass which is citrinin-free and rich in minerals, proteins and amino acids, fatty acids and dietary fiber. The Monascus product generated by the invention possesses anti-oxidation property, and shows anti-proliferation property on cancer cells via apoptosis pathway (programmed cell death).

Definition

The term “therapeutically effective amount” or “therapeutic dose” as used herein means the amount of a particular agent sufficient to provide a therapeutic benefit in the treatment or prevention of a disorder.

The terms “Monascus biomass” means a pure Monascus biomass, its extract, concentrates, mycelial powder or freeze-dried powder.

Unless otherwise defined, all technical and scientific terms used herein have the same meaning as commonly understood by one of ordinary skill in the art to which this invention belongs. Although methods and material similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

Thus, the method of the invention is characterized by a submerged fermentation for producing a pure Monascus biomass which is citrinin-free.

The method for preparing a citrinin-free Monascus biomass according to the invention includes:

providing an inoculum of Monascus purpureus;

inoculating the inoculum in a medium;

fermenting the medium under a submerged condition; and

harvesting the Monascus biomass from the medium.

The source of the Monascus strain used in the invention is well known to those skilled in the art. Culture collection centres, e.g. American Type Culture Collection, Institute of Microbiology, Academia Sinica, China, sell the cultures of Monascus species for pigment or statin production. In a preferred embodiment of the invention, Monascus purpureus strain IM 888 is used. However, in the art, this strain produces citrinin.

In the invention, an inoculum of Monascus purpureus is first prepared. The inoculum may be those well known to artisan in the art. In the prior art, conidial cultures (equivalent to spores and/or spore germlings) have been employed as inocula. For instance, the inoculum for submerged fermentative production of antibiotic penicillin and enzyme cellulase by fungi Penicillium and Trichoderma, respectively, is a conidial culture. Also, conidial inocula have been employed in producing Monascus pigments.

Though inocula (spores, conidia, spore germlings, mass of cells) in the art can be used in the invention, preferably, a homogenised mycelial culture is used as an inoculum to perform the invention. The merit lies in the convenience and ease in scaling up.

The culture is grown to a log phase (the most active growth phase) when the shortest time is taken to have the maximum fungal biomass. Rejuvenilization of the inoculum (rather than entering the stationary phase following the log phase but re-entering the brief lag and log phase) is achieved by homogenisation before inoculating the inoculum to submerged fermentation, and the fungal mycelium is fragmented into multiple growth units for inoculation. If a conidial culture is used as an inoculum, a conidial germling is one growth unit. Conidial germination frequency is not always at 100%. Also, there is not a necessity to implement an induction stage to stimulate conidiation (spore formation) usually achieved by environmental trigger in a mycelial culture. Thus the procedure is simplified.

Mycelial inoculum may be in use with mushroom cultivation. In one embodiment of the invention, a mushroom complete medium (CM) formulated by Raper & Miles (1958) is used to prepare the mycelial inoculum (Chiu & Poon, 1993).

The preparation of the inoculum has to be respected and followed strictly in order to yield optimally for the products especially in submerged fermentation. In one specific example of the invention, an inoculum culture is prepared by blending a 7-day old fully grown plate culture with 20 mL distilled water, and a mycelial homogenate is inoculated into 100 mL of CM and incubated at 32±2° C. at 200 rpm for 7 days under continuous illumination. A 7-day old culture is a culture of Monascus at the log phase. If this inoculum preparation uses an old culture, it will increase the time taken for a good yield of and heterogeneity in the inoculum culture. An old culture refers to an increasing amount of dead biomass without the benefit of increasing growth points for biomass gain. An old culture also refers to that the culture is at the stationary phase, and it will take a longer time for the target organism to juvenile in submerged fermentation.

Many media can be used for the submerged fermentation according to the invention. For example, a semi-defined broth containing 25% cane juice and 1% glutamate is used in an embodiment of the invention. The glutamate used may be monosodium glutamate or monopotassium glutamate, and preferred is monosodium glutamate, which lowers the investment cost. Another example of the fermentation medium may include 7.5% water extract of sugarcane residue (prepared by shaking 7.5 g sugar residue in 100 mL water for 2 h at 150 rpm, and the mixture was filtered to get the filtrate as the medium) supplemented with 1% monopotassium glutamate or monosodium glutamate.

The fermentation of the invention may be performed in a bioreactor which is well known to those skilled in the art. In one preferred embodiment of the invention, the fermentation is conducted at 25.0±1.0° C., with aeration and shaking as well as illumination. Fermentation at a higher temperature does not favor statin production, but fermentation at a lower temperature simply reduces growth rate and metabolic rates and does not optimize for the biomass and metabolite production. Preferably, at the beginning of the fermentation, the bioreactor is kept stationary. Fungal physiology is disturbed by blending. The fungal homogenate regenerates cell wall in the fermentation medium and become intact growth units.

In the invention, the fermentation under higher aeration and shearing force as well as illumination may help the fungus biosynthesize pigments and antioxidants. A wrapped plate culture incubated in darkness remains white in colony color, revealing that illumination triggers production of Monascus pigments. Stationary cultures yield yellowish white biomass but the red color in biomass intensifies with shaken cultures. When the shaking speed increases, the red color intensity also increases.

Also, the medium being high in C, low in P and N will affect the fungal physiology. Supplementation of glutamate is to provide a readily available nutrient source to enhance growth using C and N anabolism, shift to the biosynthesis of N-containing polypeptide pigments instead of N-free polypeptide citrinin, and shift to de novo synthesize water-soluble pigments instead of the conventional fat-soluble pigments.

After the fermentation, resultant biomass is harvested by a conventional procedure such as filtration and some smaller mycelia may be precipitated by centrifugation such as using a Beckman JZ-MI model. The biomass can then be freeze-dried for storage.

The Monascus biomass prepared in the invention contains rich source of pigments, minerals, protein and amino acids, dietary fiber and terpenes but low fat, and possesses anti-oxidation and anti-proliferation effect on cancer cell growth.

Pure Monascus biomass is fed to mice in acute and subchronic tests and proved to be safe. Aqueous extract of the Monascus biomass is effective in inhibiting proliferation of cells of prostate cancer, liver cancer and colon cancer. Lovastatin, which has recently found to be anti-cancer, is sparingly soluble in polar solvents. Both alcohol and water extracts contain anti-oxidation agents.

Monascus biomass according to the invention is thereby used as an active component of a pharmaceutical or a functional food for preventing or treating disorders associated with proliferating cancer cells in a subject.

A pharmaceutical of the invention may consist of only a Monascus biomass itself, and optionally, may further include a pharmaceutically acceptable carrier. The invention also relates to a pharmaceutical composition which generally comprises a therapeutically effective amount of a Monascus biomass and a pharmaceutically acceptable carrier.

Pharmaceuticals or pharmaceutical compositions of the invention can be formulated into dosage forms for different administration routes utilizing conventional methods, in which suitable carriers such as pharmaceutical diluents and excipients are employed. For example, they can be formulated in a capsule, a gel seal, or a tablet for oral administration. Liquid preparations for oral administration can take the form of, for example, solutions, syrups or suspensions, or they can be presented as a dry product for constitution with water or other suitable vehicle before use.

Carriers used in the invention are those commonly used in the art. Examples are, but not limited to, solid carrier such as starch, solvents such as water and alcohols.

The functional food of the invention generally comprises an amount of a Monascus biomass in need thereof. The functional food can be prepared as dietary supplements such as capsules, mini-bags, tablets or food products such as oil preparations, terpene preparations, Monascus fermented drinks, soft drinks, teas, herbal tea-bags, bakery products, sweets, snack, cakes, pudding, or confectionary.

The food also possesses other nutrients, such as proteins, carbohydrates, vitamins, minerals, amino acids and fatty acids. It also contains bioactive squalene, lovastatins and GABA.

The preventive or therapeutic dose of Monascus biomass in the treatment or prevention of proliferation-related disorders will vary with the condition to be treated and the severity of the condition to be treated. The dose, and perhaps the dose frequency, will also vary according to the age, body weight, and response of the individual patient. The nutritionist, dietitian, clinician or treating physician will know how and when to interrupt, adjust, or terminate therapy in conjunction with individual patient response.

As mentioned above, the present invention encompasses functional food comprising Monascus biomass as dietary supplements. The dietary supplements also provide means for preventing, or reducing the likelihood or experiencing, the disorders discussed above. The dietary supplements should be taken daily for at least twelve weeks and can be used permanently on a daily basis. A daily dose is from about 0.1 g to about 5.0 g; preferably about 1 to about 4 g; and most preferably about 1.2 to about 2.4 grams per day.

The invention will be further described with the following examples with reference to the drawings, which are not intended to limit the scope of the claim.

### EXAMPLES

### Example 1

### Submerged Fermentation for Monascus purpureus

Sources of Monascus purpureus

Monascus purpureus strain IM 888 was maintained at 30° C. in a mushroom complete medium (CM; Raper & Miles 1958) (Chiu & Poon, 1993).

Preparation of Inoculum

A seed culture was prepared by blending a 7-day old fully grown CM plate culture (9 cm diameter plate containing 20 mL medium) with a 20 mL distilled water, and the resultant mycelial homogenate was inoculated into a 100 mL CM and incubated at 32° C. at 200 rpm in a shaker under continuous illumination (Innova 4340 illuminated refrigerated incubator shaker, New Brunswick Scientific) for 7 days to obtain an inoculum for use in the next step.

Submerged Fermentation

A semi-defined broth containing a 25% cane juice (v/v diluted with distilled water) and 1% monopotassium glutamate by weight was prepared as a fermentation medium.

The medium was determined and found to contain: glucose 7 g/L, sucrose 12 g/L, protein 140 mg/L, phosphate 14 mg/L, 0.72 mM of asparagine and 0.06 mM of alanine [amino acid analysis and phosphate determination by the methods described below].

Medium glucose content was quantified using the dinitrosalicylic acid method (DNS) (Http://www.Engr.umd.edu/˜nsw/ench485/lab9d.htm). The composition of DNS reagent consisted of: 3,5-dinitrosalicyclic acid (7.49 g/L), NaOH (13.98 g/L), sodium potassium tartrate (216 g/L), sodium metabisulfite (5.86 g/L) and phenol (5.37 ml/L) which was first melted at 50° C. 50 μL of medium sample or glucose standard was mixed with 100 L of DNS reagent in a test tube. After boiling the mixture for 10 min and immediately cooling in an ice bath, 0.23 mL of the mixture was pipetted out and added to 1.35 mL distilled water and the absorbance at 540 nm was measured using a microplate reader (MR5000, Dynatech).

Medium sucrose content was determined after acid hydrolysis with one drop of 32% HCl to 1 mL medium sample. After 5 min at 90° C., 2 drops of 12N NaOH was then added for neutralization. Glucose content of the resulting solution was determined as stated above using the DNS method. The difference for the medium sample without acid hydrolysis and after acid hydrolysis was calculated as the sucrose content.

The fermentation medium also contained trace amounts of minerals (mg/L): Al, 2.11; Mg, 0.26; Zn, 0.22; Cu, 0.11; Fe, 0.11; Mn, 0.01; Pb, 0.06; Co, 0.02; Ni, 0.02; Cd, <0.01, and Hg, undetected (mineral contents analysed using the method described below).

The inoculum prepared above was inoculated into the fermentation medium in a bioreactor [5 L, 10 L and twin fermentor system giving a total volume of 20 L have been used; models used are: BIOSTAT B (5 L working volume, B. Braun Biotech International), BIOSTAT E (10 L working volume, B. Braun Biotech International); and Twin Fermentor system (20 L in total, each 10 L, New Brunswick)]. In all cases, the ratio of inoculum to fermentor volume was fixed at 0.5 g mycelial homogenate/L medium (fresh weight basis; equivalent to 0.05 g mycelial homogenate/L (dry weight basis)). The bioreactor was kept stationary under continuous illumination with 2 fluorescent lamps (each 60 W) at a distance of 2 m away at the first day after inoculation. The bioreactor was then set at 28° C. with aeration of 6NL/min and with shaking at 250 rpm for 5 days under continuous illumination. High aeration and shearing force as well as illumination provided as the fermentation conditions stressed the fungus to biosynthesize pigments and antioxidants as desired.

Harvest of Monascus Biomass

Monascus biomass produced was harvested by filtration first with a cheesecloth and then the smaller mycelia were precipitated by centrifugation at 12,000 rpm for 15 min using a Beckman JZ-MI model. The biomass was freeze-dried (Labconco, FREEZONE 6) for storage.

The biomass yield is 6.6890±0.8356 g/L medium (freeze-dry weight).

### EXPERIMENT 1

### Color & Nitritional Analyses

Analysis of Monascus Biomass

The biomass prepared by Example 1 was extracted with 90% ethanol at a ratio of 1:100 (w/v) for 2 h, and the extract was dried by rotary evaporation at 50° C. (BUCHI Rotavapor R-114 and BUCHI Waterbath B-480). The residue was redissolved in 1 mL methanol (HPLC grade), membrane-filtered (0.45 μm membrane, Acrodisc 4CR, PTEE) and analysed by HPLC conditions stated below.

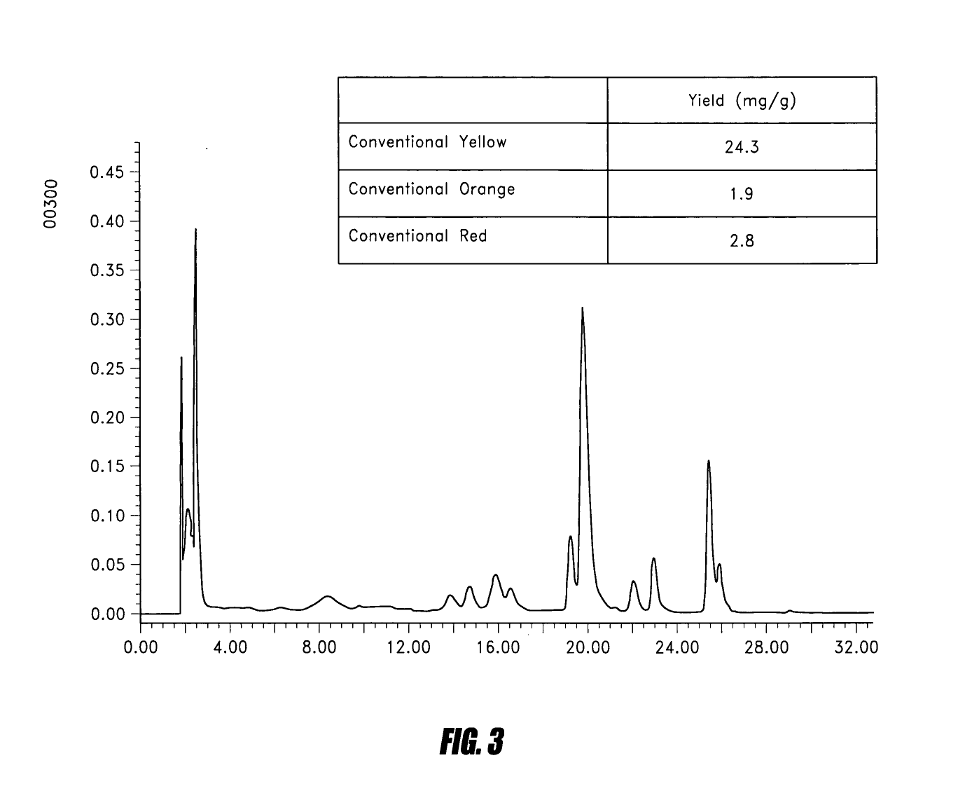

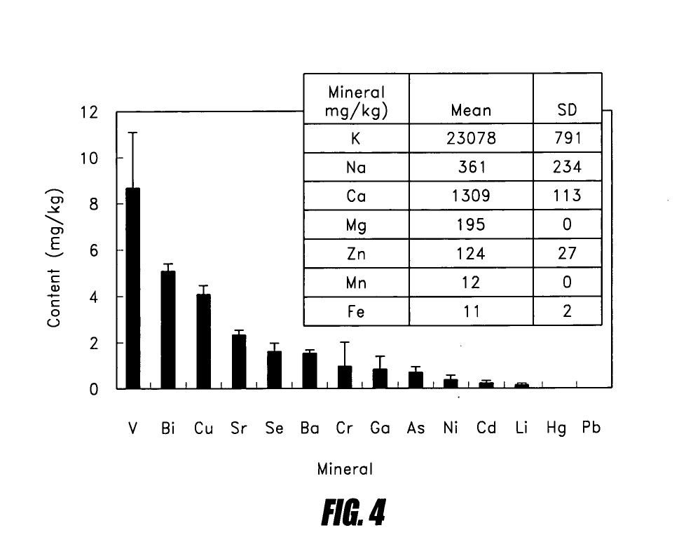

As shown in FIG. 3, there are multiple red compounds present in the alcohol extract of the Monascus biomass with absorbance at 500 nm. Only the standards for conventional pigments are available. Thus quantification is done for these conventional pigments with the following procedures.

Five gram of the freeze-dried fungal biomass was first extracted with 100 mL n-hexane overnight.

The hexane-soluble fraction was dried by rotary evaporation at 40° C. Then 20 mL of absolute ethanol were added. The alcohol phase was pipetted out and dried by rotary evaporation. After the addition of 10 mL ether, two fractions were collected: ether-soluble fraction which contained conventional orange pigments and ether-insoluble fraction which contained conventional yellow pigments.

To the hexane-insoluble residue, 100 mL of benzene were added to extract conventional red pigments overnight. The benzene-soluble fraction was dried by rotary evaporation at 40° C. Then 5 mL of absolute ethanol were added thereto. The alcohol phase was pipetted out and dried by rotary evaporation. The residue was dissolved in 1.5 mL absolute methanol (HPLC grade) and filtered (0.45 μm membrane, Acrodisc 4CR, PTEE).

The pigment standards (a gift of Dr. Sweeny, J. G. from Coca Cola, Atlanta, Ga., U.S.A.) were run in parallel. The pigment standards are not commercially available, and Dr. Sweeny prepared and provided the standards as a gift. 10 μL sample/pigment standard were injected to a high performance liquid chromatograph (HPLC) (Waters WAT027324) with a C18 reversed phase μBondapak column (300 mm×3.9 mm). The HPLC condition followed that of Lin et al. (1992) and is mentioned below. The injection temperature was at room temperature and the detection was by a tunable detector (Waters 486). The gradient separation was applied linearly from 35% to 80% aqueous acetonitrile in 19.3 min. Flow rate was kept at 1.0 mL/min. All these pigment preparations were scanned from 200 to 600 nm.

Mineral, P, C, H and N Contents

After ashing according to AOAC (1990), the metal content of the Monascus biomass prepared by Example 1 was determined with inductively coupled plasma (ICP) spectrometry (Atomscan 16 sequential Plasma Spectrometer, Thermo Jarrell Ash) or with atomic absorption spectrophotometry (Hitachi model Z-82000 series polarized Zeeman atomic absorption spectrophotometer). All glass and plastic wares used in metal determinations were acid-treated prior to use.

The C, N, and H contents of the Monascus biomass were determined with 2.000 mg of fungal powder (mortar and pestle grinded freeze-dried powder from Example 1) (AD-4 autobalance, Perkin Elmer) and placed into the CHNS/O analyser (PE 2400, Perkin Elmer). The contents of the derived carbon dioxide, nitrogen dioxide and water were then recorded.

For total P content, 0.5000 g freeze-dried sample from Example 1 was weighed into a digestion tube. Then 5 mL 69% nitric acid, 1 mL 37% HCl and 0.5 mL 98% sulphuric acid were added, and the mixture was acid-digested at a heating block (VELP DK 42/26) for 1 h at 100° C. After cooling down, the suspension was diluted to 10 mL with deionized water and filtered through Whatman No. 41 filter paper. The filtrate was further diluted to 50 mL using a 50 mL volumetric flask. Available orthophosphate or the converted orthophosphate from total P in the sample was determined by automated ion analyzer (Lachat) after extraction with (NH₄)SO₄ buffer (pH=3.0) at 150 rpm for 30 min using QuickChem method No. 10-115-01-1-A. The orthophosphate ion (PO₄²⁻) would react with ammonium molybdate and antimony potassium tartrate under acidic condition to form a complex at 37° C. This complex was further reduced by ascorbic acid to form a blue complex. The absorbance at 880 nm was measured with reference to a series of standard solution whose concentration ranged from 0-2 mg potassium dihydrogen phosphate/L. Two blanks without samples were run in parallel.

The C, N, and H contents of the Monascus biomass were summarized in Table 1. Minerals (mg/kg biomass) in the Monascus biomass include: K, 23078; Na, 361; Ca, 1309; Mg, 195; Zn, 124; Mn, 12; Fe, 11 and others such as Bi, Sr, Se, were shown in FIG. 4: Monascus biomass is a rich source of macronutrients, e.g. K, Ca and Mg but is not contaminated with heavy metals. The crude protein content is usually converted from the total N content by multiplying a factor of 6.25 according to AOAC (1990). The N content of Monascus biomass reaches 10.69±0.15% (referring to Table 1). This crude protein is then predicted as 66.8%. The conversion factor of 6.25 for fungi is a reference (AOAC, 1990) but this factor actually changes with different physiological stages of fungus Pleurotus pulmonarius ranging from 4-6 (Chiu et al., 1998). Edible mushrooms generally regarded as a high protein food next to milk and soybean have 20-30% crude protein (calculated using the multiplication with the conversion factor). Thus Monascus biomass is a high protein product.

TABLE 1

| | Mean | SD |
|---|---|---|
| C (%) | 41.46 | 0.45 |
| H (%) | 5.93 | 0.05 |
| S (%) | 0.02 | 0.01 |
| N (%) | 10.69 | 0.15 |
| Total P (mg/g) | 5.54 | 0.09 |
| Total minerals (mg/g) | 29 | 1 |
| Ergosterol (mg/g) | 3.26 | 0.83 |
| Terpene (%) | 1.83 | 0.38 |
| Fat (%) | 3.84 | 0.84 |
| Water-soluble polysaccharide (%) | 2.91 | 0.49 |
| Chitin (%) | 12.69 | 0.81 |
| Dietary fiber (%) | 49.26 | 1.47 |
| Total amino acids (mg/g) | 0.23 | 0.00 |

Total and Free Amino Acids Analysis

One gram of the freeze-dried Monascus biomass prepared in Example 1 was grinded to 0.2 mm diameter in size using a Cyclotec (Tecator) 1093 sample mill and then prepared following the manufacturer's instruction and Gehrke et al. (1985). 0.1000 g of the sample was subjected to vapor phase hydrolysis by 7 mL of 6N HCl (Sigma H0636) at 110° C. for 24 h. After drying in a Speedvac Concentrator, 5 mL of Na-S buffer (Cat no. 94303-0803, Beckman) was added to dissolve the residue.

For free amino acids, 1.0000 g of the freeze-dried sample was immersed in 10 mL of autoclaved water and shaken for 1 h. The supernatant after centrifugation was collected and processed similarly. One mL of filtered sample (0.45 μm Nylon Acrodisc filter, Gilman) was then examined by an amino acid analyzer (Beckman 7300 system, CA, U.S.A.). A commercially available mixture of seventeen naturally occurring amino acids (cat. No. 338088, Beckman) and GABA (cat No. A5835, Sigma) were analyzed in parallel and used as the standard.

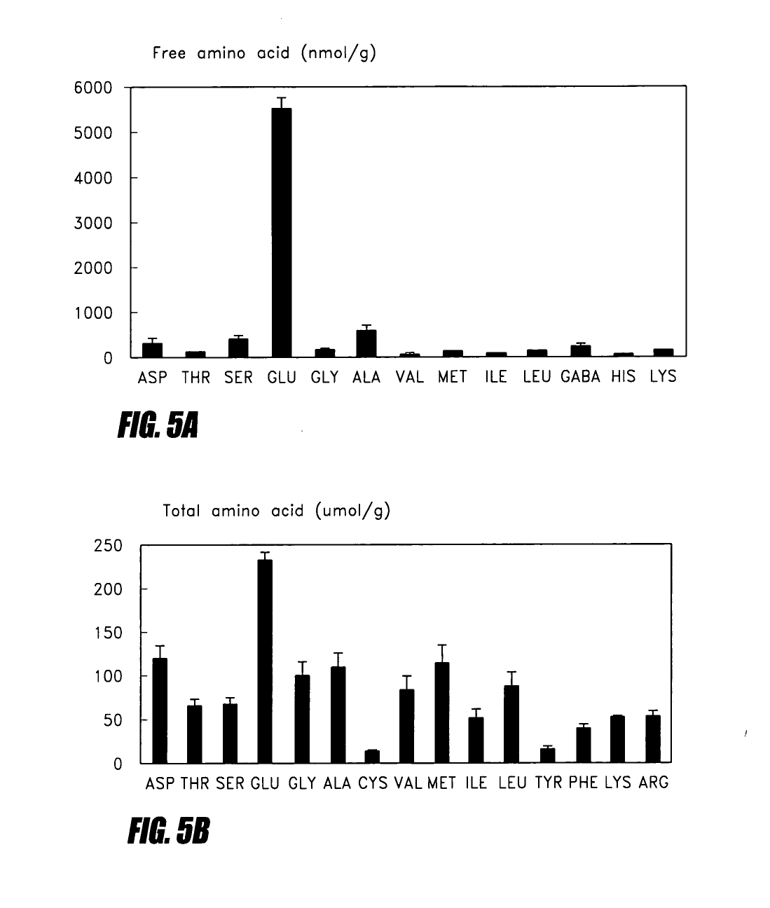

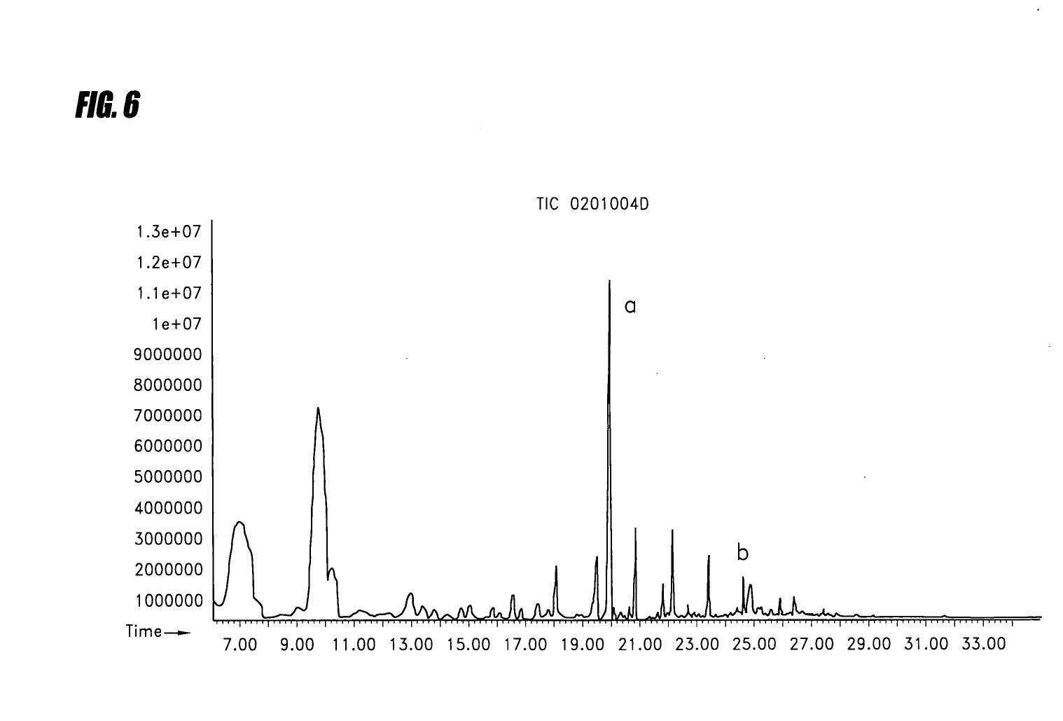

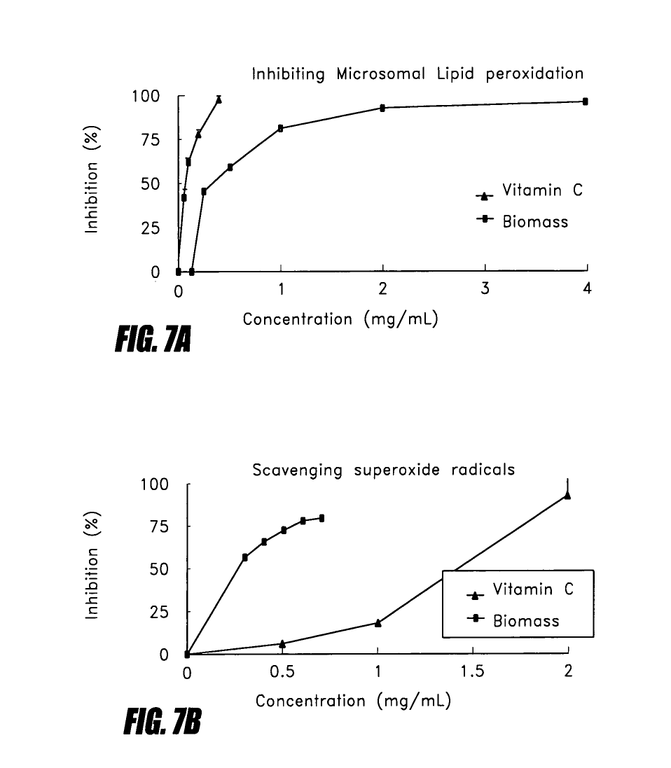

The total amino acid content accounts for 0.23% of Monascus biomass (Table 1). Glutamate is the major amino acid in the free pool and in the total pool, as shown in FIG. 5. The modified amino acid GABA was detected in the free pool at a content of 221±63 nmol/g. Fifteen amino acids were detected in the total pool. ARG, CYS, TYR and PHE were not detected in the free pool while HIS was not detected in the total pool (FIG. 5). The extreme high glutamate in free pool is explained by the glutamate added to the fermentation medium as C- and N-sources. Monascus biomass of the invention provides a balanced spectrum of amino acids essential for human nutrition.

Dietary Fiber Content

Assay was performed with a commercial kit (Sigma TDFAB-2) using the AOAC method (1990). The freeze dried biomass of Example 1 was homogenized into powder by a miller (Tecator, 1093 cyclotec sample mill). 1.0000 g of the sample was placed in a 250 mL Erlenmeyer flask. 25 mL of phosphate buffer (1.4 g of anhydrous Na₂HPO₄ and 8.4 g of NaH₂PO₄ in 1000 mL of water; pH 6.0) were added to the flask and followed by 0.05 mL of α-amylase (Sigma A3306). All samples were covered with an aluminum foil and placed in a boiling water bath with gently shaking. The flask was incubated for 15 min when the internal temperature of the flask reached 95° C. Then the sample was cooled to room temperature, and the pH was adjusted to 7.5±0.2 with 5 mL of 0.275N NaOH. 50 mg of protease (Sigma P3910) were dissolved in 1 mL of phosphate buffer, and 0.1 mL of protease was pipetted into a sample. The mixture was then incubated at 60° C. with continuous agitation for 30 min. After cooling, the pH of the sample was adjusted to pH4.0-4.6 with 5 mL of 0.325M HCl. 0.15 mL of amyloglucosidase was added and incubated at 60° C. for 30 min. Four volumes of 95% ethanol were added, and the solution was let to stand overnight at room temperature. After centrifugation at 10,000×g for 15 min, the precipitate was collected and washed with three 20 mL portions of 78% ethanol and two 10 mL portions of 95% ethanol by centrifugation. The precipitate was dried in a preweighed crucible in a 105° C. oven. After cooling in a desiccator, the crucible was weighed and recorded as ‘residue+crucible weight’. The dietary fiber content was calculated as follows:

$$\frac{\begin{matrix}Final weight of a crucible - Initial weight of a crucible - \\ net weight of a blank\end{matrix}}{Weight of a sample} \times 100 \%$$

The dietary fiber content of Monascus biomass was: 49.26±1.47% (Table 1). This content is comparable to mushrooms, a high fiber food. Much research work suggests that dietary fiber may prevent cancer, diabetes, heart disease and obesity. In plants, dietary fiber refers to complex polysaccharides: water-insoluble fibers, e.g. cellulose, hemicellulose and lignin, and water-soluble fibers, e.g. gum and pectin. The fungal dietary fiber includes the glycogen granules in cells, water-soluble polysaccharides and insoluble cell wall polysaccharides including chitin. Cell wall polysaccharides and water-soluble polysaccharides of many fungi are found to have anti-cancer properties and immunomodulatory properties.

Chitin Assay

0.1 g of a freeze-dried sample of Example 1 was undergone alkaline hydrolysis by 4 mL 1N NaOH (Sigma). The mixture was then boiled at 120° C. for 15 min. After cooling, a pellet in the mixture was collected by centrifugation at 14,000 rpm for 3 min (Eppendorf MiniSpin Plus 5453), and washed with distilled water by centrifugation until the solution became colorless. The solution was measured at 260 nm and 280 nm to ensure that the absorbance readings were zero, meaning that no more protein eluted out. The pellet was then demineralized using 2N HCl in a solid/solvent ratio of 1:10 (w/v). The reaction mixture was heated at 95° C. overnight and the supernatant was collected by centrifugation at 14,000 rpm for 3 min. One mL of 12N NaOH was added to the supernatant and the precipitate formed was collected by filtration with an oven-dried and pre-weighted GF/C filter (a glass filter, Whatman). Then the filter was dried in an oven at 105° C. for 24 h. The percentage of chitin content in the sample (Monascus biomass) was calculated according to the following equation,
Chitin content (%)=(W_{f}−Wᵢ)/Wₛ×100%
where W_{f} is the dry weight (g) of chitin residue with the filter, Wᵢ is the dry weight (g) of the filter, and Wₛ is the dry weight (g) of the sample.

The chitin content in the Monascus biomass was 12.69±0.81% (Table 1). Chitin constitutes to the cell wall polysaccharide and the insoluble dietary fiber.

Determination of Water-Soluble Polysaccharide by Anthrone Method

One mL of Anthrone solution (0.1 g of anthrone dissolved in 76% sulphuric acid to a volume of 100 mL) was added to 0.1 mL of a sample from Example 1 at one of the following concentrations (1 to 40 mg/mL). The solution was heated at 100° C. for 15 min. The mixture was cooled to room temperature and the absorbance was measured at 620 nm. Glucose was used as the reference for constructing a standard curve. Water-soluble polysaccharide content in a sample was measured as glucose-equivalent content.

The water-soluble polysaccharide content in the Monascus biomass was 2.91±0.49% (Table 1). Water-soluble polysaccharides of many mushroom species are found to have anti-cancer and immunomodulatory bioactivities. The water extract of Monascus biomass for bioactivity assay also contains water-soluble polysaccharides.

Fat Content and Fatty Acid Profile

Fat content in a sample from Example 1 was determined by the solvent extraction method using a Soxhlet extraction apparatus (AOAC, 1990). Total fatty acids were extracted by saponification and determined by ester derivation and then followed by analysis using GC system for cellular fatty acid profiling (MIDI). Fatty acids were liberated by cell lysis using a saponifcation reagent [Sodium hydroxide (Riedel-de Haën, Germany), 45 g; methanol (HPLC grade, Mallinckrodt), 150 mL; ultrapure water, 150 mL]. Methyl esters of fatty acids (FAMEs) were formed by a methylation reagent [6N hydrochloric (Mallinckrodt), 325 mL; methanol (HPLC grade, Mallinckrodt), 275 mL]. FAMEs were then extracted from the aqueous phase to the organic phase by a mixture of methyl tert-butyl ether (MTBE) and 85% n-hexane. Organic extracts were washed by a base wash [Sodium hydroxide (Riedel-de Haën, Germany), 10.8 g; ultrapure water, 900 mL] before gas chromatography analysis with Hewlett-Packard 6890.

The fat content of Monascus biomass was 3.84±0.84% (Table 1), and thus Monascus biomass is a low-fat product. However, the fatty acid profile reveals that there is a balanced spectrum of saturated and unsaturated fatty acids as shown in Table 2. Oleic acid (C18 fatty acid) is the predominant fatty acid. This is the first report on the fatty acid profile of Monascus.

TABLE 2

Relative
Abundance (a.u.)

| Chemical Formula | Fatty Acids | Mean | SD |
|---|---|---|---|
| 10:0 | Capric Acid | 458 | 18 |
| 12:0 | Lauric Acid | 381 | 17 |
| 14:0 | Myristic Acid | 921 | 184 |
| 15:0 | | 539 | 471 |
| 16:1 w9c | | 990 | 185 |
| 16:1 w7c/15 iso 2OH | | 1971 | 385 |
| 16:0 | Palmitic Acid | 52968 | 12798 |
| 17:1 w8c | | 1959 | 532 |
| 17:0 | Margaric Acid | 1599 | 525 |
| 18:2 w6, 9c/18:0 Ante | | 101009 | 25243 |
| 18:1 w9c | Oleic Acid | 155116 | 40035 |
| 18:1 w7c | Asclepic Acid | 385 | 666 |
| 18:0 | Stearic Acid | 27351 | 8055 |
| 19:0 Iso | Isoarachidic Acid | 1184 | 399 |
| 18:0 2OH | | 1152 | 1001 |
| 20:0 | Arachidic Acid | 483 | 419 |

Terpene Analysis by GC-MS

A homogenized freeze-dried sample (0.1 g, of Example 1) was saponified with 0.5 mL of saturated aqueous KOH in 8 mL of absolute ethanol at 85° C. for 30 min. Unsaponifiable matters were extracted with 20 mL of cyclohexane, and then the hydrolysate was diluted with 12 mL of water. The extract was dried under rotary evaporation at 50° C. and weighed. Then the residue was re-suspended in 1 mL of diethyl ether (HPCL grade), and membrane-filtered (0.45 μm membrane, Acrodisc 4CR, PTEE). The sample was analyzed by gas chromatograph/mass spectrophotometry (Shimadzu GCMS-QP5050A) on HP-5MS crosslinked 5% PH ME siloxane column (0.25 mm×30 m×25 μm film thickness). A temperature profile used was: injector temperature started at 60° C. and ramped at 8° C./min to 300° C. and maintained at 300° C. for 10 min.

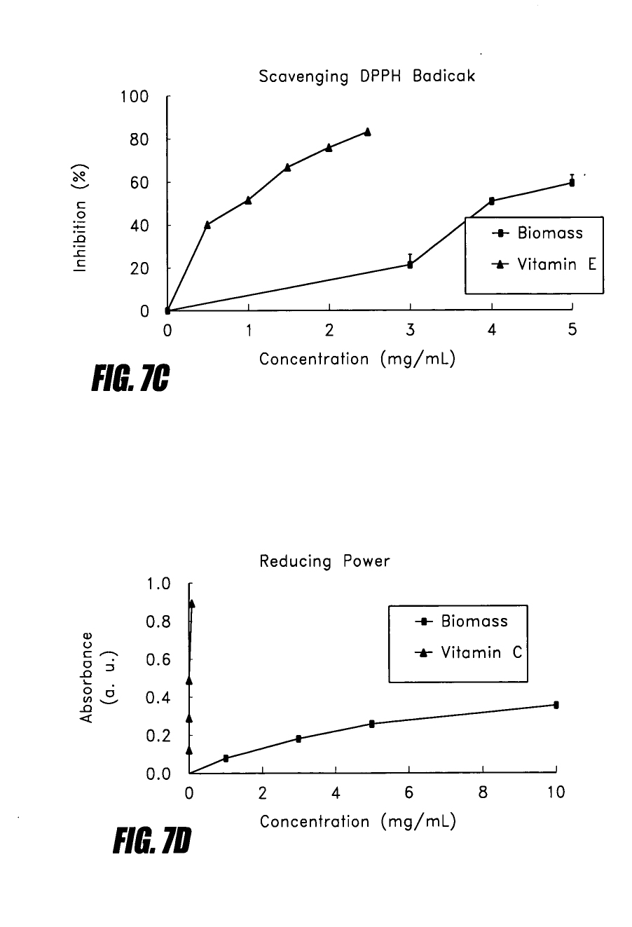

This is the first report on the terpene content of Monascus. Terpenes of mushrooms are reported to have anti-cancer property. The terpene content in the Monascus biomass was 1.83±0.38% (Table 1). In the GC chromatogram, both squalene and ergosterols are resolved as distinct peaks as shown in FIG. 6. The ergosterol content was 3.26±0.83 mg/g (=0.326±0.083%) while squalene content was 1.80±0.17 mg/g (=0.180±0.017%). Both squalene and ergosterol show anti-proliferation effect on cancer cells.

### EXPERIMENT 2

### Bioactivity Test

1. Antioxidation Capacities

A freeze-dried sample of Example 1 was weighed and made up to 100 mg/mL in distilled water. The solution was autoclaved at 121° C. for 15 min. The supernatant was harvested after centrifugation at 4° C. was stored at −20° C. A serial dilution was then made with the stock aqueous extract into a series of increasing concentrations of the extracts up to 100 mg/mL solution. For the assay of the scavenging of DPPH radicals, a methanolic extract (membrane-filtered; 0.45 μm membrane, Acrodisc 4CR, PTEE) (replacing the solvent water with methanol and procedure stated in the first sentence) was used instead.

Assay for Inhibition of Lipid Peroxidation

Normal rats were sacrificed by cervical dislocation. The liver of the rats was rapidly homogenized in 0.25M of an ice-cold sucrose solution and the resultant solution was centrifuged at 12,000×g for 20 min at 4° C. The supernatant obtained was centrifuged at 15,000×g for 60 min at 4° C. The microsomes were washed using ice-cold 0.15% KCl, and then stored at −20° C.

The microsomes (200-300 μg/mL) were incubated at 37° C. for 60 min with the extract sample of Monascus biomass at variable concentrations, 10 μM FeSO₄ and 0.1 mM ascorbic acid in 1.0 mL of a phosphate buffered saline (PBS, 0.25M, pH7.4). The product of microsomal lipid peroxidation was malondialdehyde (MDA). The reaction was stopped by 20% (w/v) trichloroacetic acid (TCA, 1.0 mL) and 0.67% (w/v) 2-thiobarbituric acid (TBA, 1.5 mL) in succession, and the solution was then heated at 100° C. for 15 min. After centrifugation of precipitated protein, the color reaction of MDA-TBA complex was detected at OD 532 nm. Vitamin C and PBS were used as positive and negative controls, respectively.

The inhibition rate (%) was calculated using the following formula:
Inhibition rate (%)=(A−A₁)/A×100%

where A was the absorbance (OD₅₃₂) of the negative control, and A₁ was that of the tested sample.

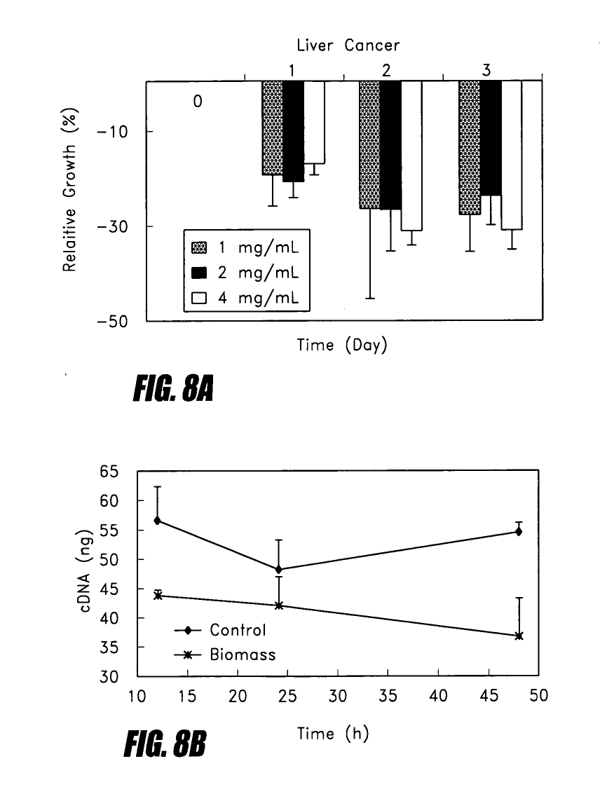

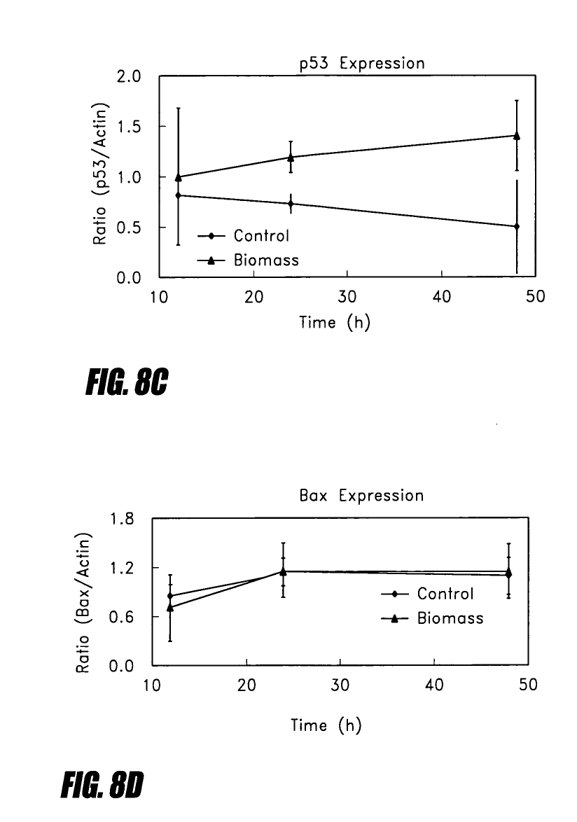

FIG. 7a shows the result. The effective concentration of Monascus extract in giving 50% inhibition of microsomal lipid peroxidation was 0.4 mg/mL while it took 0.1 mg Vitamin C/mL to give a similar effect. Thus the Monascus extract which is not a pure compound but shows strong anti-oxidation capacity to inhibit lipid peroxidation.

Assay for Scavenging of Superoxide Radicals

Superoxide radicals were generated in a PMS-NADH [phenazin methosulfate (PMS)-β-nicotinamide adenine dinucleotide (reduced from, NADH)] system by oxidation of NADH and assayed by the reduction of nitroblue tetrazolium (NBT). The superoxide radicals were generated in 3 mL of Tris-HCl buffer (16 mM, pH8.0), which contained 78 μM NADH, 50 μM NBT, 10 μM PMS and Monascus extracts at different concentrations to be tested. The color reaction between superoxide radicals and NBT was detected at OD 560 nm. L-ascorbic acid was used as a positive control. The inhibition rate (%) was calculated using the following formula:
Inhibition rate (%)=(A−A₁)/A×100%

where A was the absorbance of a negative control (distilled water), and A₁ was the absorbance of the tested sample.

As showed in FIG. 7b, Monascus extracts showed a stronger anti-oxidation capacity in this scavenging of superoxide radicals than the positive control, vitamin C. The effective concentrations giving 50% inhibition were: 0.25 mg/mL for Monascus extract and 1.5 mg/mL for Vitamin C.

Assay for Scavenging of DPPH (2,2-diphenyl-1-picrylhydrazyl) Radicals

1.8 mL of a methanolic solution of DPPH (6×10⁻⁵M 2,2-diphenyl-1-picrylhydrazyl) was added to 0.2 mL of a methanolic Monascus extract of variable concentrations (prepared as described above). The absorbance of the reaction mixture was measured at 515 nm exactly 30s after the DPPH solution was added. Vitamin E and methanol were used as positive and negative controls, respectively.

The inhibition rate (%) was calculated using the following formula:
Inhibition rate (%)=(A−A₁)/A×100%

where A was the absorbance of the negative control, and A₁ was that of the tested sample.

As with the case of superoxide radicals, this anti-oxidation capacity was tested basing on the scavenging ability. However, Monascus extract performed less well in comparison to the positive control (FIG. 7c). The effective concentrations to give 50% inhibition were: 1 mg/mL for Vitamin E and 4 mg/mL for Monascus extract. In comparison to other mushroom extracts, Monascus extract performed better.

Reducing Power

2.5 mL of 1% potassium ferricyanide [K₃Fe(CN)₆] was added to 1.0 mL of an aqueous Monascus extract of variable concentrations (prepared as stated above). The resultant mixture was incubated at 50° C. for 20 min. 2.5 mL of trichloracetic acid (TCA, 10%) were added to the mixture. 2.5 mL of the resultant solution were mixed with 2.5 mL of distilled water and 0.5 mL of FeCl₃ solution (0.1%). The absorbance was measured at 700 nm. Increased absorbance of the reaction mixture indicated an increase of reducing power. Vitamin C was used as a positive control in this experiment.

The reducing power of Vitamin C, the positive control, is very high acting at the range of less than 1 mg/mL, as shown in FIG. 7d. In contrast, the Monascus extract acted at mg/mL level and showed a plateau at 10 mg/mL onwards. As the response curve of Monascus extract does not fit to a straight line with the increasing amount of Monascus extract, this might indicate that there are multiple compounds present in the extract interacting to give such a curve relationship. Overall speaking, Monascus extract does show reducing power.

Four different assay systems were practiced to assess the anti-oxidation capacities of Monascus extracts, as showed from FIGS. 7a to 7d. All demonstrates that the Monascus biomass of the invention has a significant effect of antioxidation. The scavenging ability in removal of superoxide radicals of Monascus extract is even better than Vitamin C, a commercial anti-oxidant.

2. Anti-Proliferative Effect on Cancer Cells

Human cancer cells [lines: HB-8064 (liver cancer), HB-8065 (liver cancer), HTB-22 (breast cancer), CCL-221 (colon cancer), CRL-5803 (lung cancer), CRL-1435 (prostate cancer); ATCC] were maintained in a Dulbecco's modified Eagle's medium (DMEM) supplemented with a 10% fetal bovine serum (FBS), 100 mg/L of streptomycin and 100 IU/mL of penicillin. The cancer cells were seeded to a 96-well plate (Nunc, Denmark), each at a density of 1.5×10³/cm², and incubated for 1 day at a CO₂ incubator under a humidified atmosphere of 5% CO₂ at 37° C. before supplementing with aqueous extracts of a freeze-dried sample from Example 1 at variable concentrations by a method as described in the “Anti-Oxidation Capacities”.

Cell Proliferation Measured by MTT Assay

Cell proliferation and viability of a sample of Example were monitored by measuring a change of the tetrazolium salt using a cell proliferation (MTT) kit (Roche Molecular Biochemicals). According to the manual of the kit, the treated cancer cells were incubated with a tetrazolium dye (MTT) labelling solution. Metabolic active cells cleaved MTT to form formazan with a strong red absorption band at 550-618 nm. Thus, after 4 h of incubation, the cells were lyzed, and the purple formazan crystals were solubilized for detection at 570 nm.

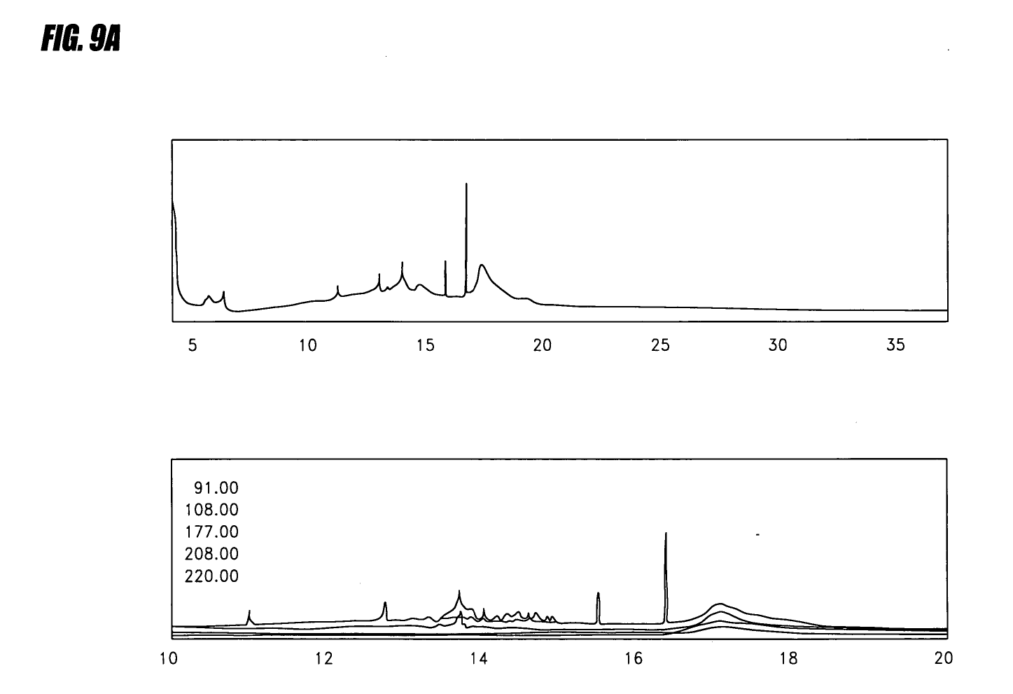

Among the human cancer cell lines examined, Monascus extracts showed anti-proliferation effect on colon cancer cells, liver cancer cells and prostate cancer cells but were ineffective against breast cancer cells and lung cancer cells. FIG. 8a shows that Monascus extract caused an inhibition on proliferation (i.e. negative growth) of liver cancer cells. Such anti-proliferation effect started at day 1 and persisted to day 3. Monascus extracts at the same dose showed higher inhibition effect on liver cancer cells than colon cancer cells and prostate cancer cells. Thus the molecular mechanism of the anti-proliferation effect was examined further with liver cancer cells (cell line HB-8065, ATCC).

Action Mechanism of Anti-proliferation on Liver Cancer Cells by RT-PCR

Total RNAs were extracted from the cultured cells using TRIzol (invitrogen) according to the manufacturer's instructions. RNA pellets were dissolved in diethyl pyrocarbonate-treated water, and the concentrations and purity were calculated from OD₂₆₀ and OD₂₈₀. First strand cDNA synthesis (invitrogen) was performed from total RNA (2 μg) in a reaction mixture containing 500 oligo (dT) and 1 mM dNTP mix. Tubes were heated (65° C., 5 min). The following reagents were added: 5×first-strand buffer, 0.1M dithiothreitol, a recombinant ribonuclease inhibitor (40 U) and a Superscript II RNase H reverse transcriptase (200 U) (invitrogen). cDNA synthesis was performed for 50 min at 42° C.

Comparing the cDNA yields from the control and treated cells with Monascus extract of Example 1, the Monascus extract decreased the cDNA yields with time. This trend of decreasing cDNA yield with time shows a linear relationship (r²=0.9887 where r is the correlation coefficient) (FIG. 8b). In contrast, no such relationship is detected with the control. Thus extracts of Monascus biomass inhibit cell proliferation (FIG. 8a) and reduce the total RNA amounts (which is quantified as cDNA yields by reverse transcriptase reaction) of the liver cancer cells (FIG. 8b).

In order to examine whether Monascus extracts of the invention inhibit proliferation of liver cancer cells through regulation of gene expression, the expression profiles of two genes were examined: tumor suppressor gene p53 whose product functions to promote cell cycle arrest and apoptosis (programmed cell death). Gene Bax, whose product is an apoptotic protein, is only induced or enhanced at apoptosis. Gene p53 was upstream while gene Bax is downstream in the apoptosis pathway. The expression level of the target gene was normalized to the expression level of a housekeeping gene, actin, so that the differential effects of trigger, enhancement or inhibition of gene expression in the liver cancer cells are revealed. The procedures are as follows:

PCR reactions were carried out in 50 μL of a mixture containing 100 ng of cDNA, 100 μM of a primer shown in Table 3, 50 mM KCl, 20 mM Tris-HCl (pH8.4 at 25° C.), 1.5 mM MgCl₂, 0.01% (v/v) Tween 20, and a 0.2 mM each of dATP, dCTP, dGTP and dTTP, using 1 U of Taq polymerase (invitrogen).

TABLE 3
Primer sets for the Following Genes Used

| | | | Amplified |
|---|---|---|---|
| | | | Product |
| Genes | Forward Primer | Backward Primer | (bp) |
| Bax | 5′-cagctctgagcagatcatgaagagc-3′ | 5′-gcccatcttcttccagatggtgagc-3′ | 538 |
| p53 | 5′-ctgaggttggctctgactgtacca-3′ | 5′-ctcattcagctctcggaacatctc-3′ | 220 |
| Actin | 5′-ggagcaatgatcttgatctt-3′ | 5′-cctctgggcatggagtcct-3′ | 180 |

The following conditions were used: 95° C., 3 min followed by 24-31 cycles of denaturation (95° C., 50s), annealing (61° C., 50s), and extension (72° C., 50s). Amplification products were analyzed by ethidium bromide-agarose gel electrophoresis using 2% (w/v) agarose gels and the bands captured under UV illumination. All primer sets generated clean products of the predicted sizes. Products were analyzed by scanning densitometry and normalized to a housekeeping gene actin.

FIG. 8c shows that the contrasting effects on the differential gene expression profiles of p53 along incubation time in the absence or presence of the extracts of Monascus biomass. In the control, p53 expression level dropped with time. This means that the liver cancer cells proliferated and increased in population size with time for the control. When linear regression analysis is performed with this set of data, a decreasing straight line relationship can be described by an equation of: Y=−0.0089X+0.929 with r²=0.9959.

In contrast, extracts of Monascus biomass enhanced the expression of p53 with time showing an increasing trend with time. This also matches with the fact that the population size of liver cancer cells decreased with time (FIG. 8a). This increasing trend fits to a straight line relationship which is described by an equation of: Y=0.0107X+0.898 with r²=0.9751.

When another gene Bax was under examination, different results were obtained. FIG. 8d shows the result. The expression levels of Bax in both treatment and control were similar along incubation time.

Thus, extracts of Monascus biomass inhibited proliferation of liver cancer cells. The total RNA amount was also reduced by the Monascus treatment. The inhibition on proliferation was via the enhanced expression of the tumor suppressor gene p53 and not gene Bax.

3. Chemical Determination of Lovastatin by GC-MS

5 g of a freeze-dried sample as prepared in Example 1 were extracted with 100 mL chloroform for 2 hr at 100 rpm. The chloroform phase was collected. 100 mL of fresh chloroform was added to the residue for another extraction. Both chloroform phases were combined and dried by rotary evaporation. The residue was redissolved in 1 mL of methanol (HPLC grade), membrane-filtered (0.45 μm, Acrodisc 4CR, PTEE) and analysed by GCMS. An authentic standard lovastatin was run in parallel for confirmation of the retention time and quantification.

The Monascus biomass produced by submerged fermentation as described in Example 1 contained 0.244±0.022 mg/g of lovastatin (=0.0244±0.0022%). A commercial red rice purchased from the market was found to contain 0.098±0.055 mg/g of lovastatin which reflects a lower content and a larger variance.

### EXPERIMENT 3

### Food Safety Assay

Detection for Citrinin by High Performance Liquid Chromatography and GC-SIM

An extraction of citrinin (syn. monascidin A) was prepared according to the procedures of Blanc et al. (1995). In brief, 10 g of biomass from Example 1 were first extracted with 500 mL acetonitrile for 3 hr. After addition of an equal volume of water and acidification to pH4.5 with 2N H₂SO₄, chloroform (equal volume to that of water) was added. The lower chloroform phase was collected and dried under rotary evaporation. The residue was redissolved in 1 mL of absolute methanol (HPLC grade), membrane-filtered and analyzed by HPLC (Waters HPLC system). A c18 reversed phase μBondapak column (300 mm×3.9 mm; Waters WAT027324) was operated at ambient temperature with a tunable absorbance detector (Waters 486) set at 250 nm. Elution was performed by a gradient of 15% to 65% aqueous methanol within 12 min at a flow rate of 1.0 mL/min. The detection sensitivity using authentic citrinin standard (Sigma C1017) at 1 μg/mL absolute methanol (HPLC grade) gave 0.054a.u. resolved at 14.5 min.

Also, the purified sample as prepared above was injected to a GCMS-QP5050A gas chromatograph mass spectrometer (SHIMADZU) with a 3000×0.25 mm HP-5MS gas column (Agilent Technologies), AOC-20i auto injector (SHIMADZA), and AOC-20s autosampler (SHIMADZU). A temperature profile was set: injector temperature started at 70° C. and ramped at 21° C./min to 280° C. and maintained at 280° C. for 10 min. The standard citrinin was run in parallel and resolved by selected fragment ions (m/z): 91, 105, 177, 205 and 220, at two retention times: 15.5 and 16.4 min (Shu & Lin, 2002).

Extraction and purification of citrinin were performed with the produced Monascus biomass from Example 1 did not show a citrinin peak by HPLC although a citrinin authentic standard did. The experiment was repeated for ten times, and the identical result was obtained. The medium after fermentation was freeze-dried and processed for detection of citrinin too. However, no citrinin was detected. When the same strain of Monascus purpureus was fermented using a Hajjaj et al. medium (1999 & 2000), citrinin was detected as the result reported in Hajjaj et al. (1999 & 2000).

In order to confirm the result, the produced Monascus biomass from Example 1 was artificially spiked with citrinin, and the whole extraction, purification and analysis procedure was followed. Citrinin was extracted at an efficiency of 95±3%. Therefore, the failure in detecting a peak of citrinin in Monascus biomass might be because of the absence in biosynthesis of citrinin by M. purpureus at the fermentation conditions.

In order to confirm the absence of citrinin, another analysis method is needed. A commercial immunoassay system for detecting citrinin in food is available. However the immunoassay system is based on conjugated colorimetric reaction which is not applicable as the Monascus pigments will be extracted out at the same time, and pigments will interfere such detection.

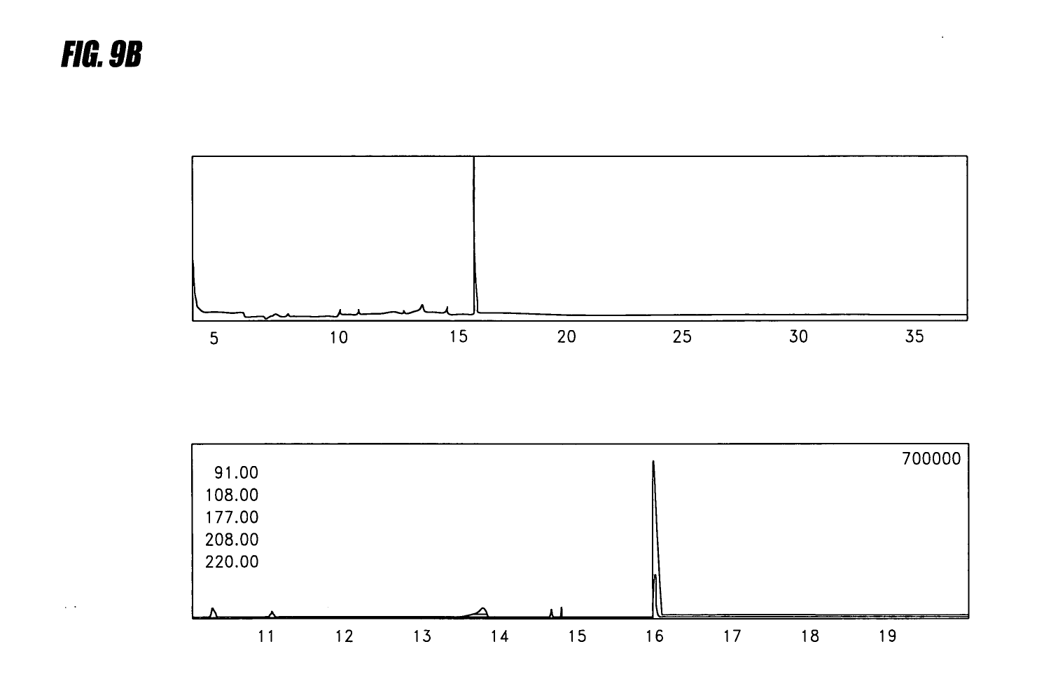

Rather, a detection method using GC-SIM was adopted. Using a gas chromatography (GC) for analysis has the advantage over HPLC in terms of sensitivity (mg/mL and ug/mL for HPLC and GC, respectively). However, citrinin is fragmented by the temperature profile. Therefore two peaks of citrinin breakdown products with the characteristic fragmented ions were resolved as shown in FIG. 9a. The Figure is generated by a citrinin authentic standard. One μg citrinin/mL yielded a peak at 15.5 min with an ion abundance of 10,000 and another peak at 16.4 min with an ion abundance of 30,000. These two peaks with the characteristic ion fragment profiles (5 ion fragments) are visible and distinct. When 10 g of Monascus biomass produced by Example 1 were used to extract for citrinin, and the preparation was analysed by GC-SIM, no corresponding peaks of citrinin at both retention times were detected (FIG. 9b). Thus citrinin is not produced by Monascus under the production process in Example 1 as analysed by the biochemical methods.

As citrinin is toxic, and the LD₅₀ (lethal dose causing decrease of 50% population) of citrinin for mouse is: 35 mg/kg. A bioassay system was also carried out. Also, red rice (Monascus-fermented rice) is regarded as a GRAS (generally regarded as safe) product. The toxicity assays using mouse model also test whether Monascus biomass is safe for consumption or not.

Acute and Subchronic Toxicity on Mice

Male ICR mice were obtained from The Animal House, The Chinese University of Hong Kong. Ten mice were housed together in a stainless steel cage (37 cm×27 cm×12 cm) with a 12hr dark-12hr light cycle. The mice were fed with rodent chow (Streges Bates, Ridley Agri product, Australia) and water. All the mice were acclimatized for 2 weeks before use. The mice were fasted overnight before experimentation. The treatment groups were administered with 7.5 g (d.w.) of homogenized Monascus biomass from Example 1 per kg body weight. A control group was given with an equal volume of ultrapure water. For testing acute toxicity this was a once-only treatment. For subchronic toxicity tests the animals were fed with the homogenized Monascus biomass or with ultrapure water daily for eight weeks.

All animals were observed closely after administration of the Monascus biomass. The general appearance of the animals was noted and compared with the control group. After the experimental period all the mice were sacrificed by inhalation of ether. Autopsies were performed. Fresh weights of heart, kidney, liver, lung and spleen were measured. Blood samples were drawn by syringe from the posterior vena cava for further analysis. Serum glutamate-pyruvate transaminase (GPT) and glutamate-oxaloacetate transaminase (GOT) levels were assayed using commercial kits (Sigma 505) and following the supplier's instructions. Serum urea nitrogen was assayed using a Sigma BUN (endpoint) kit.

Mice appeared and behaved normally when the control group was compared to the treatment group with Monascus biomass. No abnormality was detected in both the acute and subchronic toxicity assays. The body weight gains in both the control and treatment groups were similar. At the end of the experiment, the animals were sacrified. The organ to body weight ratios showed no significant difference between the control and the treatment groups in the acute and subchronic toxicity assays by student T test (p<0.05). Similarly, the serum enzymes GOT, GPT and urea nitrogen of the control and treatment groups in both acute and subchronic toxicity assays were the same (student T test, p<0.05).

Citrinin is a nephrotoxin, heptatoxin and carcinogen. If liver is damaged, the serum GOT and GPT will be increased. If kidney is damaged, the serum urea nitrogen will be increased. Citrinin has a LD₅₀ of 35 mg/kg. 7.5 g powdered Monascus biomass/kg was orally administrted to a mouse. All the mice in the treatment groups of the acute and subchronic toxicity assays were normal as the control groups. Thus, the Monascus biomass does not contain citrinin as revealed by the bioassay method. This bioassay method also confirms the biochemical assay method for detection of citrinin in Monascus biomass. The mouse model assays also reflect the safety of Monascus biomass for consumption.

It is intended that all subject matters contained in the above description, shown in the drawings, or defined in the appended claims, be interpreted as descriptive and illustrative, and not in a limiting sense. Modifications and variations of the invention without departing from the spirit of the invention are possible in light of the teachings disclosed herein. It is therefore understood that the invention can be practiced otherwise than as specifically described within the scope of the appended claims.

### REFERENCES

1. AOAC (1990) Official Methods of Analysis of the Association of Official Analytical Chemists. Vol. I and II. 15 edn. Association of Official Analytical Chemists, Inc.
2. Blanc, P. J., Laussac, J. P., Le Bars, J., Le Bars, P., Loret, M. O., Pareilleux, A., Prome, D., Prome, J. C., Santerre, A. L. and Goma, G (1995) Characterization of monascidin A from Monascus as citrinin. International Journal of Food Microbiology 27: 201-213.
3. Chiu, S. W. & Poon, Y. K. (1993) Submerged production of Monascus pigments. Mycologia 85, 214-215.
4. Chiu, S. W. Chan, Y. H., Law, S. C., Cheung, K. T. & Moore, D. (1998) Cadmium and manganese in contrast to calcium reduce yield and nutritional value of the edible mushroom Pleurotus pulmonarius. Mycological Research 102, 449-457.
5. Gehrke, C. W., Wall, L. L. Sr, Absheer, J. S., Kaiser, F. E. & Zumwalt, R. W. (1985) Sample preparation for chromatography of amino acids: acid hydrolysis of proteins. Journal of the Association of Official Analytical Chemists 68, 811-821.
6. Hajjaj, H., Blanc, P. J., Groussac, E., Goma, G, Uribelarrea, J. L. & Loubiere, P. (1999) Improvement of red pigment/citrinin production ratio as a function of environmental conditions by Monascus ruber. Biotechnology and Bioengineering 64, 497-501.
7. Hajjaj, H., Klaebe, A., Goma, G, Blanc, P. J., Barbier, E. & Francois, J. (2000) Medium-chain fatty acids affect citrinin production in the filamentous fungus Monascus ruber. Applied and Environmental Microbiology 66, 1120-1125.
8. Raper, J. R. & Miles, P. G (1958) The genetics of Schizophyllum commune. Genetics 438, 530-546.
9. Shu, P. Y., & Lin, C. H. (2002) Simple and sensitive determination of citrinin in Monascus by GC-selected ion monitoring mass spectrometry. Analytical Sciences 18, 283-287.

What is claimed is:

1. A method for preparing a Monascus biomass, including:
providing an inoculum of a Monascus purpureus;
inoculating the inoculum in a medium;
fermenting the medium under a submerged condition; and
harvesting the Monascus biomass from the medium,
wherein the Monascus biomass is free of citrinin.

2. The method of claim 1, wherein the Monascus purpureus is Monascus purpureus strain IM 888.

3. The method of claim 2, wherein the inoculum is a mycelial culture.

4. The method of claim 3, wherein the inoculating is performed under illumination.

5. The method of claim 1, wherein the medium is a semi-defined broth.

6. The method of claim 2, wherein the fermenting is firstly kept stationary and then shaken.

7. The method of claim 6, wherein the fermenting is preformed under aeration and illumination.

8. A Monascus biomass prepared by:
providing an inoculum of a Monascus purpureus;
inoculating the inoculum in a medium;
fermenting the medium under a submerged condition; and
harvesting the Monascus biomass from the medium,
wherein the Monascus biomass is free of citrinin.

9. The biomass of claim 8, wherein the Monascus purpureus is Monascus purpureus strain IM 888.

10. The method of claim 9, wherein the inoculum is a mycelial culture.

11. The method of claim 10, wherein the inoculating is performed under illumination.

12. The method of claim 8, wherein the medium is a semi-defined broth.

13. The method of claim 9, wherein the fermenting is firstly kept stationary and then shaken.

14. The method of claim 13, wherein the fermenting is preformed under aeration and illumination.

15. A pharmaceutical for preventing or treating a cell proliferation-associated disorder in a subject comprising an effective amount of a Monascus biomass of claim 8.

16. The pharmaceutical of claim 15, wherein the disorder is cancer.

17. The pharmaceutical of claim 16, wherein the cancer is hepatoma or colon carcinoma or prostate carcinoma.

18. A method for preventing or treating a cell proliferation-associated disorder in a subject comprising administrating a therapeutically effective amount of a Monascus biomass of claim 8 to the subject in need thereof.

19. The pharmaceutical of claim 18, wherein the disorder is cancer.

20. The pharmaceutical of claim 19, wherein the cancer is hepatoma or colon carcinoma or prostate carcinoma.

21. A functional food comprising a Monascus biomass of claim 8.


SEQUENCE LISTING (6 sequences)

SEQ ID NO: 1 - DNA, 25 bp, Artificial Sequence
  Forward primer for Bax gene
cagctctgagcagatcatgaagagc

SEQ ID NO: 2 - DNA, 25 bp, Artificial Sequence
  Backward primer for Bax gene
gcccatcttcttccagatggtgagc

SEQ ID NO: 3 - DNA, 24 bp, Artificial Sequence
  Forward primer for p53 gene
ctgaggttggctctgactgtacca

SEQ ID NO: 4 - DNA, 24 bp, Artificial Sequence
  Backward primer for p53 gene
ctcattcagctctcggaacatctc

SEQ ID NO: 5 - DNA, 20 bp, Artificial Sequence
  Forward primer for actin gene
ggagcaatgatcttgatctt

SEQ ID NO: 6 - DNA, 19 bp, Artificial Sequence
  Backward primer for actin gene
cctctgggcatggagtcct